(19) United States
(12) Patent Application Publication
(10) Pub. No.: US 2017/0100505 A1
(43) Pub. Date: Apr. 13, 2017

(54) ABSORBENT PRODUCT COMPRISING A MICROBE-INHIBITING COMPOSITION

(71) Applicant: SCA Hygiene Products AB, Göteborg (SE)

(72) Inventors: Petter Andreassen, Goteborg (SE); Vania Lizet Romero Gomez, Zapopan (MX)

(73) Assignee: SCA HYGIENE PRODUCTS AB, Göteborg (SE)

(21) Appl. No.: 15/317,174

(22) PCT Filed: Jun. 10, 2014
(86) PCT No.: PCT/SE2014/050702

§ 371 Date: Dec. 8, 2016

(51) Int. Cl. A61L 15/44; A61F 13/511; A61L 15/40; A61L 15/18; A61L 15/34

(52) U.S. Cl. CPC A61L 15/44; A61L 15/18; A61L 15/34; A61L 15/40; A61F 13/51113; A61L 2300/102; A61L 2300/30; A61L 2300/404; A61F 2013/51117

(57) ABSTRACT

An absorbent product such as a diaper, pant diaper, adult incontinence protector, incontinence pad, sanitary napkin or panty liner including a liquid permeable top sheet is disclosed. A microbe-inhibiting composition is applied on at least a portion of the top sheet. The microbe-inhibiting composition includes a lipid carrier, Aloe Vera, and zinc oxide.

### CROSS-REFERENCE TO PRIOR APPLICATION

This application is a §371 National Stage Application of PCT International Application NO. PCT/SE2014/050702 filed Jun. 10, 2014, which is incorporated herein in its entirety.

### TECHNICAL FIELD

The present disclosure relates to an absorbent product such as a diaper, sanitary napkin, panty liner, incontinence protector, tampon, or the like, that includes a top sheet including a microbe inhibiting composition.

### BACKGROUND

The urogenital area is a sensitive region with a delicate balance of moisture, fat and microorganisms, which is easily disturbed and which may cause discomfort. For example, the balance may be disturbed by extensive cleaning, the use of antibiotics or antifungal agents, and the use of absorbent products that may dry out the skin and mucous membranes. It is known that the administration of different substances to the urogenital area, such as prebiotics, probiotics, skin soothing substances etc., may improve the balance in the urogenital area and/or prevent discomfort.

The urogenital area harbors a complex microbial ecosystem including many different bacterial species. The microbial ecosystem of a healthy individual can be disturbed by the use of for example antibiotics. Also, microorganisms may spread from the anus to the urogenital area, thereby causing infections. This results in a disturbance of the normal microbial flora and leaves the individuals susceptible to microbial infections that cause vaginitis, urinary tract infections and ordinary skin infections.

Microorganisms commonly associated with these kinds of infections belong to the genera Escherichia, Enterococcus, Pseudomonas, Proteus, Klebsiella, Streptococcus, Staphylococcus, Gardnerella and Candida.

Staphylococcus aureus is the most common cause of minor skin infections, such as boils or abscesses, as well as more serious post-operative wound infections. Treatment involves drainage and this is usually sufficient for minor lesions, but antibiotics may be given in addition when the infection is severe and the patient has fever.

One way to reduce the problems with the kind of infections described above is to have a good personal hygiene. However, excessive use of cleaning agents not only decreases the amount of harmful microbes, but can harm the beneficial microbial flora, again render it susceptible for pathogenic species to colonize and cause infections.

In view of the prior art there is still a need for absorbent products with an improved inhibitory effect on unwanted microorganisms.

### SUMMARY

It is desired to provide an absorbent product with an improved inhibitory effect on unwanted microorganisms. It is further desired to improve the distribution of a microbe-inhibiting composition in the final products so that an efficient microbe-inhibiting system can be obtained without impairing the absorption properties in the final product.

The above defined problem is solved by an absorbent product, such as an absorbent product in the form of a diaper, adult incontinence protector, incontinence pad, sanitary napkin or panty liner including a liquid permeable top sheet, wherein a microbe-inhibiting composition is applied on at least a portion of the top sheet and wherein the microbe-inhibiting composition includes a lipid carrier, Aloe Vera and zinc oxide. The microbe inhibiting composition of zinc oxide and Aloe Vera gives an unexpected synergistic effect in inhibition of pathogenic microorganisms, and an absorbent product including this microbe inhibiting composition has an enhanced probiotic and/or prebiotic effect.

Extracts from Aloe Vera are widely used in the cosmetics and alternative medicine industries, being marketed as variously having rejuvenating, healing, or soothing properties. Aloe vera is for example used on facial tissues where it is promoted as a moisturizer and anti-irritant to reduce chafing of the nose. Cosmetic companies commonly add derivatives from Aloe vera to products such as makeup, tissues, moisturizers, soaps, sunscreens, incense, shaving cream, or shampoos. A review of academic literature notes that its inclusion in many hygiene products is due to its “moisturizing emollient effect”. The Aloe Vera may be added to the lipid carrier as a liquid or in a dry state.

Zinc oxide is commonly used as a sun blocking ingredient. Zinc oxide has a long history of safe use. It is not irritating and compatible with sensitive skin. In fact, zinc oxide is a skin protectant and anti-irritant, and is widely used in treating various forms of dermatitis/skin irritation. The zinc oxide which is suitable for use includes those inorganic white and yellowish white powders that conform to the formula ZnO.

According to one embodiment, the zinc oxide is in the form of a powder. Powders are very difficult to handle in dry processes due to dusting problems. Therefore, an advantage by mixing the powder of the zinc oxide with a lipid carrier is that contamination of both the process equipment and the products with the powder of the zinc oxide is avoided.

According to one embodiment, the lipid carrier is selected from petroleum derived lipid, synthetic lipid, animal or plant derived lipid and is in the form of a fat, oil, or a wax or a mixture thereof.

The lipid carrier may be petrolatum. Petrolatum is a petroleum based hydrocarbon with carbon numbers mainly higher than 25. Synonym to petrolatum is petroleum jelly, white petrolatum, soft paraffin or multi-hydrocarbon and the Brand name Vaseline is often used. Petrolatum is hydrophobic and is originally promoted as a topical ointment for its healing properties. It is widely used as a skin protectant, and is commonly used in the cosmetic skin care area.

According to one embodiment, the concentration of the lipid carrier is 80.0-96.5 weight percent, the concentration of the zinc oxide is 1.5-10.0 weight percent, and the concentration of the Aloe Vera is 2.0-10.0 weight percent.

According to one embodiment, the concentration of the lipid carrier is 88.0-96.0 weight percent, zinc oxide is 1.5-4.0 weight percent, and Aloe Vera is 2.5-8.0 weight percent.

According to yet another embodiment, the concentration of the lipid carrier is 92.0 weight percent or less, zinc oxide is at least 2.5 weight percent, and Aloe Vera is at least 5.5 weight percent. The concentration of the lipid carrier may be 82.0-92.0 weight percent, zinc oxide may be 2.5-8.0 weight percent, and Aloe Vera may be 5.5-10 weight percent.

According to yet another embodiment, the micro-inhibiting composition is a substantially homogenous mix of the zinc oxide, Aloe Vera, and the lipid carrier.

According to one embodiment, said microbe-inhibiting composition is applied in an intermittent pattern with first regions coated with the microbe-inhibiting composition and second regions free of the microbe-inhibiting composition. The top sheet, for example the nonwoven in baby diapers, should be pervious to liquids and has therefore usually been prepared to be hydrophilic. The further finishing with a hydrophobic microbe-inhibiting composition may therefore reduce or significantly impair the transportation of liquid through the web into the absorbing materials. It is therefore advantageous, that the microbe-inhibiting composition covers only 1-20% of the total surface area of the top sheet, or 1-10% of the total surface area of the top sheet, or 1-5% of the total surface area of the top sheet.

According to one embodiment, the pattern of the first regions with the microbe-inhibiting composition is at least two stripes extending generally parallel in relation to each other in the longitudinal direction of the absorbent product. By using a lipid carrier, which is hydrophobic, it is also possible for the microbe-inhibiting composition to function as a liquid barrier making it possible to steer the liquid in a certain direction. According to one embodiment, the microbe-inhibiting composition is applied to the top sheet in stripes in the longitudinally direction of the absorbent product. The longitudinal stripes across the length of the diaper will function as liquid barriers in the longitudinal direction of the absorbent product and thereby decrease the risk of the liquid to be spread in the cross direction of the product.

The pattern of the first regions with the microbe-inhibiting composition may be two stripes extending generally parallel in the longitudinal direction of the absorbent product wherein the length in the transverse direction of the absorbent product from the longitudinal centre line of the absorbent product, is the same for each stripe. The length in the transverse direction of the absorbent product from the longitudinal centre line to each stripe may be between 7 mm to 15 mm. It is advantageous that the region in the wetting zone, is free from microbe-inhibiting substance since the lipid carrier makes the microbe-inhibiting composition hydrophobic.

According to yet another embodiment, the pattern of the first regions with the microbe-inhibiting composition is two stripes extending generally parallel in the longitudinal direction of the absorbent product and the distance between the two stripes in the transverse direction of the product is at least 16 mm, or at least 18 mm.

According to one embodiment, each stripe has a width of 1.0-8.0 millimeter, or a width of 2.0-5.0 millimeter.

The absorbent product, according to one embodiment, has a liquid impermeable backsheet, a liquid pervious topsheet and an absorbent core enclosed there between.

### DESCRIPTION OF THE DRAWING

FIG. 1 is a plan view of a diaper according to an embodiment.

### DETAILED DESCRIPTION OF PARTICULAR EMBODIMENTS

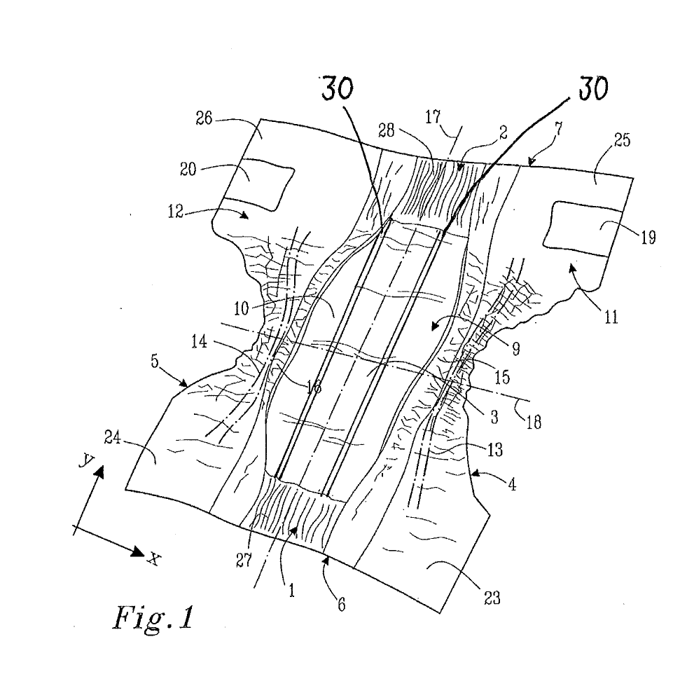

As an example of an absorbent product, a diaper will be described. The diaper has a longitudinal direction—the y-direction—with a longitudinal centre line 17 and a transverse direction—the x-direction—with a transverse centre line 18. In FIG. 1, a diaper is shown which includes a front and a rear end portion 1,2, a crotch portion 3 lying there between, which, in use of the diaper, is intended to be brought between the legs of the user. The diaper further has longitudinal edges 4,5, a front transverse edge 6 and a rear transverse edge 7, a lower liquid impermeable back sheet 8, a first liquid permeable top sheet 10 and an absorbent core 9 placed there between. Further, the diaper possesses longitudinal leakage barriers 15,16 which run substantially parallel to the longitudinal centre line 17 of the diaper and near the longitudinal edges of the absorbent core 9. The leakage barriers 15,16 contain elastic and in the figure are wrinkled together at least in their centre portions.

Side flaps 11,12 extend at the sides outside the leakage barriers 15,16 and possess at least one longitudinal elastic elements 13,14 in the crotch portion 3 along their free edges. The elastic elements 13,14 serve as leg elastic upon use of the article and provide an extra leakage barrier.

The diaper also includes fastening means 19,20 (folded towards the topsheet of the diaper in FIG. 1) in the form of hook-and-loop type fastening means; in alternative embodiments, tape with glue or other similar means may also be used. The diaper may also possess reception surfaces for said fastening means (not shown in the Figures) which are specially adapted for said fastening means.

The front 23, 24 and rear 25, 26 end regions of the side flaps 11,12 are seen at the front 1 and rear end portion 2 of the diaper. The diaper shown in FIG. 1 has front and rear waist elastic 27,28 along at least a part of the front 23,24 and rear 25,26 end regions of the side flaps 11,12. As shown in FIG. 1, the waist elastic 27, 28 can be located on both transverse edges 6,7 of the diaper. Front and rear waist elastic 27, 28 extends along roughly a third of the length of each transverse edge 6,7. In other embodiments, only a rear waist elastic may be present. Waist elastic may also exist which extends entirely along both transverse edges of the absorbent article.

The absorbent core 9 may include cellulose fibres, with mixing of superabsorbent particles or superabsorbent fibres. However, the absorbent core may be constructed of any suitable standard material which is usually present in absorbent cores for absorbent articles such as diapers, pant-diapers, incontinence shields, panty liners and the like. The absorbent core 9 may be constructed of more than one layer of absorbent material. Absorbent cores usually contain layers of wadding, receiving and distribution layers, to be able to rapidly take away released liquid from the liquid-receiving top sheet 10. Each of the layers including the absorbent core may contain superabsorbents.

The liquid-impervious back sheet 8 can comprise or consist of a liquid-impermeable plastic film, a nonwoven sheet treated with liquid-resistant material, or some other flexible material layer which has the ability to resist liquid penetration. It is usually an advantage if the liquid-impermeable back sheet 8 is breathable, i.e. allows the passage of water vapour through the sheet 8.

The liquid-permeable top sheet 9 may include a nonwoven sheet of e.g. polyethene fibres, polypropene fibres or mixtures thereof, perforated films or tissue layers.

In FIG. 1, the microbe-inhibiting composition is applied to the top sheet in two stripes 30, extending generally parallel in the longitudinal direction of the diaper. The stripes 30 in FIG. 1 have a width of 2-5 millimeters. Each stripe is located 5 to 20 mm from the longitudinal centre line 17 and the length from the longitudinal centre line 17 can be the same for each stripe. It is advantageous that the region in the longitudinal centre of the diaper, the wetting zone, is free from microbe-inhibiting substance since the lipid carrier makes the microbe-inhibiting composition hydrophobic. The microbe-inhibiting composition in FIG. 1 is covering 1-5% of the total surface area of the top sheet.

It is also possible to have the microbe-inhibiting composition applied to the top sheet in more than two stripes, or in another pattern. The microbe-inhibiting composition may also be applied on elastic members, belts etc., which during use of the product is in contact with the skin of the wearer via for example the liquid pervious top sheet.

### EXPERIMENTS

The bacterial growth of Staphylococcus aureus, E. coli and Aspergillus niger was measured on three different nonwoven materials. The method used was the Antibacterial Activity Assessment of Textile Materials: Parallel Streak Method, AATCC Test Method 147-2004, which is a method to determine antibacterial activity of diffusible antimicrobial agents on treated textile materials.

### Tested Materials

1. Nonwoven comprising carrier substance of petrolatum and Aloe Vera.

2. Nonwoven comprising carrier substance of petrolatum and zinc oxide

3. Nonwoven comprising carrier substance of petrolatum, zinc oxide and Aloe Vera.

The nonwoven is made of polypropylene and has a surface weight of 12.5 gsm and the whole surface of the nonwoven material is covered with the microbe-inhibiting composition. The amount of Aloe Vera in the microbe-inhibiting composition for material 1 is 5.5 weight percent and the amount of petrolatum is 94.5 weight percent. The weight percent of zinc oxide in the microbe-inhibiting composition for material 2 is 2.5 and the petrolatum is 97.5 weight percent. The weight percent of Aloe Vera in the microbe-inhibiting composition for material 3 is 5.5, the weight percent of zinc oxide in the microbe-inhibiting composition for material 3 is 2.5 and the weight percent of petrolatum is 92.

### Procedure

Dispense sterilized nutrient agar by pouring 15 ml into each standard flat bottomed petri dish. Allow agar to gel firmly before inoculating. Prepare inoculum by transferring 1.0 ml of 24 broth culture into 9.0 ml of sterile distilled water contained in a test tube. Mix well using appropriate agitation. Using a 4 mm inoculating loop, load one loopful of the diluted inoculum and transfer to the surface of the sterile agar plate by making five streaks 60 mm in length, spaced 10 mm apart covering the central area of a standard petri dish without refilling the loop. Gently press the test nonwoven transversely across the five inoculum streaks to ensure intimate contact with the agar surface. Incubate at 37 degree Celsius for 24 hours.

### Evaluation

Examine the incubated plates for interruption of growth along the streaks of inoculum beneath the nonwoven and for a clear zone of inhibition beyond its edge. The average width of a zone of inhibition along a streak on either side of the nonwoven is calculated using the following equation:

W=(T−D)/2

where:
W=width of clear zone in inhibition in mm
T=total diameter of nonwoven and clear zone in mm
D=diameter of the nonwoven in mm
Result—Width of clear zone of inhibition in mm

| Material | S. aureus | E. coli | A. niger |
|---|---|---|---|
| 1. | 1 | 1 | 0 |
| 2. | 4 | 3 | 1 |
| 3. | 4 | 4 | 2 |

The width of the clear zone of inhibition of E. coli and A. niger was longer for material 3 than for material 1 or material 2. The microbe-inhibiting composition on material 3 comprised petrolatum, zinc-oxide and Aloe Vera. So, an unexpected increased inhibition was obtained when a combination of zinc oxide and Aloe Vera was applied to the nonwoven material compared to only Aloe Vera or only zinc oxide.

1. An absorbent product comprising a liquid permeable top sheet and a microbe-inhibiting composition applied on at least a portion of the top sheet, wherein the microbe-inhibiting composition comprises a lipid carrier, Aloe Vera, and zinc oxide.

2. The absorbent product according to claim 1, wherein the zinc oxide is in the form of a powder.

3. The absorbent product according to claim 1, wherein the lipid carrier is selected from the group consisting of petroleum derived lipid, synthetic lipid, and animal or plant derived lipid, and the lipid carrier is in the form of a fat, an oil, or a wax or a mixture thereof.

4. The absorbent product according to claim 1, wherein the lipid carrier is petrolatum.

5. The absorbent product according to claim 1, wherein the concentration of the lipid carrier is 80.0-96.0 weight percent, the concentration of the zinc oxide is 1.5-10.0 weight percent, and the concentration of the Aloe Vera is 2.0-10.0 weight percent.

6. The absorbent product according to claim 1, wherein the concentration of the lipid carrier is 82.0-92.0 weight percent, the concentration of the zinc oxide is 2.5-8.0 weight percent, and the concentration of the Aloe Vera is 5.5-10.0 weight percent.

7. The absorbent product according to claim 1, wherein the micro-inhibiting composition is a substantially homogenous mix of the zinc oxide, Aloe Vera, and the lipid carrier.

8. The absorbent product according to claim 1, wherein said microbe-inhibiting composition is applied in an intermittent pattern with first regions coated with the microbe-inhibiting composition and second regions free of the microbe-inhibiting composition.

9. The absorbent product according to claim 1, wherein the microbe-inhibiting composition is covering 1-20% of the total surface area of the top sheet.

10. The absorbent product according to claim 1, wherein the microbe-inhibiting composition is covering 1-10% of the total surface area of the top sheet.

11. The absorbent product according to claim 1, wherein the microbe-inhibiting composition is covering 1-5% of the total surface area of the top sheet.

12. The absorbent product according to claim 8, wherein the pattern of the first regions with the microbe-inhibiting composition are at least two stripes extending generally parallel in relation to each other in the longitudinal direction of the absorbent product.

13. The absorbent product according to claim 12, wherein the pattern of the first regions with the microbe-inhibiting composition are two stripes extending generally parallel in the longitudinal direction of the absorbent product, and wherein the distance between the two stripes in the cross direction of the product is at least 16 mm.

14. The absorbent product according to claim 12, wherein each stripe has a width of 1.0-8.0 millimeter.

15. The absorbent product according to claim 12, wherein each stripe has a width of 2.0-5.0 millimeter.
